(19) United States
(12) Patent Application Publication
(10) Pub. No.: US 2008/0139891 A1
(43) Pub. Date: Jun. 12, 2008

(54) Devices and methods for communicating medical information

(75) Inventors: Ronald Scott Whitehead, Atlanta, GA (US); Joaquin Aguirre, Duluth, GA (US); Juan F. Rodriguez, Atlanta, GA (US)

Correspondence Address: MOAZZAM & ASSOCIATES, LLC;MATTERS FOR CINGULAR WIRELESS II, LLC, 7601 LEWINSVILLE ROAD, SUITE 304, MCLEAN, VA, 22102, US

(73) Assignee: Cingular Wireless II, LLC

(21) Appl. No.: 11/585,972

(22) Filed: Oct. 25, 2006

(51) Int. Cl. A61B 5/00

(52) U.S. Cl. 600/300

(57) ABSTRACT

Devices and methods are disclosed which relate to the sharing of medical information securely to comply with HIPAA. A medical device is disclosed that takes medical readings and sends them, along with other pertinent medical information, to a secure server where it is stored in a database. In case of an emergency, the device also connects the user to an expert where the expert can recall pertinent medical information from a database. This database can store everything including height and weight, allergies and current medications, a history of problems and conditions, and even the instantaneous heart beat of the subject.

### BACKGROUND OF THE INVENTION

1. Field of the Invention

The present invention relates to the sharing of medical information securely to comply with HIPAA. More specifically, the present invention relates to devices and methods that take medical readings and send them, along with other pertinent medical information, to a secure server where it is stored in a database; and can also connect to an expert for emergency assistance.

2. Background of the Invention

Today there are many health statistics that are important to the average individual. Not just vital statistics like pulse and temperature, but records indicating vaccinations, allergies, and blood type, which are not only useful in some certain daily circumstances, but can become crucial in an emergency situation. However, since the enactment of The Health Insurance Portability and Accountability Act (HIPAA), certain precautions must be taken to keep this information between the patient and his or her doctor.

Emergency situations require not only the necessary information about a subject, but now also require advanced equipment. Diabetics wear necklaces and bracelets to alert those aiding in their rescue that they are diabetic, should the worst happen, and AIDS victims are now doing the same. However, there is much more information that, if known to a potential rescuer at the time, can be very beneficial and possibly life-saving, such as information about allergies or other diseases. As for the equipment, many buildings and other public facilities now have emergency defibrillators. While this machine is very useful at the right time, it has only one purpose. A more universally useful device could do so much better if it were in every building and public facility.

Emergency situations could also benefit from on-demand medical readings of a subject. The first thing a trained professional checks is pulse and breathing, but an untrained person may not know how to check for those. In some cases this wouldn't matter, because an untrained person would not know how to interpret the subject anyway, but if that person had proper instruction, those readings could become crucial to the subject's life.

Information in the form of on-demand medical readings is not just helpful in an emergency situation, but can have benefits at any time. Specially conditioned people must check their status from time to time. Diabetics must check their blood/sugar level, people with hypertension must check their blood pressure, and some elderly people have a number of things to check on periodically. Current technology can aid people with this burden, but they must take it upon themselves to act on the results. If the data were automatically communicated elsewhere, then a third party could monitor these medical readings, and act accordingly.

In a pertinent example, when soldiers engage in battle they will undoubtedly be hurt or wounded in some way, possibly mortally. Medical units will run out in an effort to save those who are wounded. The medical personnel rely on their own eyes to guide them to the wounded, but this surely isn't the best method. If the medical readings of these soldiers were monitored by the base then the medical units could concentrate on the mortally wounded first, and rescue those with only minor injuries last.

The current state of the art has wrist monitors for simple medical readings such as pulse, temperature, blood pressure, and blood/sugar. These have been in use by patients of various necessities because of the need for constant monitoring.

GPS units can give a vector, a position and a velocity, for itself, using a satellite system created and maintained by the United States Government. GPS, also known as NAVigation System Timing and Ranging Global Positioning System, or NAVSTAR GPS, has been available for public use since 1996. GPS, when first installed, gave a position accurate up to 15 meters, or 50 feet. Since 2000, a system known as the Wide-Area Augmentation System, or WAAS, has decreased the margin of error to 2 meters, or 6 feet. GPS units have become increasingly popular since their debut. Many new devices come installed with GPS units and some feature navigation systems.

Many new cellular telephones have built-in GPS units for locating in case of emergency. Cellular communication is another area of increasing technology and availability. At the end of 2005, a CTIA study showed that 69% of the United States population subscribed to a cellular telephone service. Cellular telephones work on a network of receiving towers that communicate with a central location. These towers are constantly being built in an effort to create a seamless network across the globe. Not only is this market more popular, but the technology is growing as well. Cellular companies have now opened data pipelines for delivering broadband internet straight to its customers phones.

Now there is a system that can determine position in real-time, a network that can deliver high-speed information, and many devices for taking on-demand medical readings. However, to date, there has never been a technology that combines these other existing technologies in a unique and novel way to create a device that will take on-demand readings and alert the proper expert in case of an emergency, and establish a communication connection to that expert.

### SUMMARY OF THE INVENTION

The present invention is a portable communication device for transmitting secure medical information. This device can take medical readings and transmit them through a secure connection to a base server where information is stored in a database, and/or delivered straight to an expert, depending on the situation. The readings are transmitted from a communication device either on-board or in communication with the device. The location of the device can also be detected through a locator either on-board or in connection with the invention.

Furthermore, the present invention works with current forms of communication including, but not limited to, radio, cellular and satellite. The invention can either have the communication device onboard, or in communication with it. It can be in wired or wireless communication with the invention using technology such as BLUETOOTH, infrared or others. The present invention can use any form of digital encryption to secure data sent to the base server and prevent unauthorized access.

In one exemplary embodiment of the present invention, the device is used in a plurality of buildings and public places, placed behind glass labeled, “In Case of Emergency Break Glass”. These devices are capable of taking some readings, mainly pulse, breathing, and temperature, and are capable of delivering a location. These can come in use in many emergency situations such as traumatic injury. A trained or untrained person may take the device from behind its glass encasing. A wristband will be connected to the device which should be placed on the subject's wrist immediately. A connection is instantly made with the server as the device takes the subject's pulse and temperature. An expert answers the call and instructs the user on how to rescue the subject. The expert reads the location and calls the appropriate emergency vehicle.

In another exemplary embodiment of the present invention, the device is made to be worn at all times. It can be manufactured to take only certain readings, and with or without a locator. This embodiment can be used for people with certain special conditions, such as diabetes which would include a blood/sugar monitor. Nursing homes can have their patients wear these to monitor conditions and locations of the patients, granting them more freedom to move around. Soldiers in the battlefield can wear more advanced forms of these that detect more vital signs and work in communication with the radios they already carry.

In yet another exemplary embodiment of the present invention, the device is made for use by a doctor who works abroad, perhaps in third world countries, or other places where state-of-the-art medical equipment is scarce. This device can be made to take simple readings like the emergency model, or more advanced readings like the soldier model. If the doctor comes across a patient, he or she can use this device to take the necessary readings and connect him with an expert anywhere in the world using satellite communication. The expert can then assist the doctor with whatever is needed.

### BRIEF DESCRIPTION OF THE DRAWINGS

FIG. 1 shows a diagram of the flow of data between the medical communication device and the base server according to an exemplary embodiment of the present invention.

FIG. 2 shows a medical communication device including a wrist strap for biological readings, a small screen, an onboard cellular communication device, and a speakerphone according to an exemplary emergency embodiment of the present invention.

FIG. 3 shows a medical communication device comprising a wrist strap for biological readings, and wired or wireless connectivity to a communication device according to another exemplary embodiment of the present invention.

FIG. 4 shows a medical communication device including a screen for input of medical information to be transmitted and in connection with a satellite communication device according to yet another exemplary embodiment of the present invention.

### DETAILED DESCRIPTION OF THE INVENTION

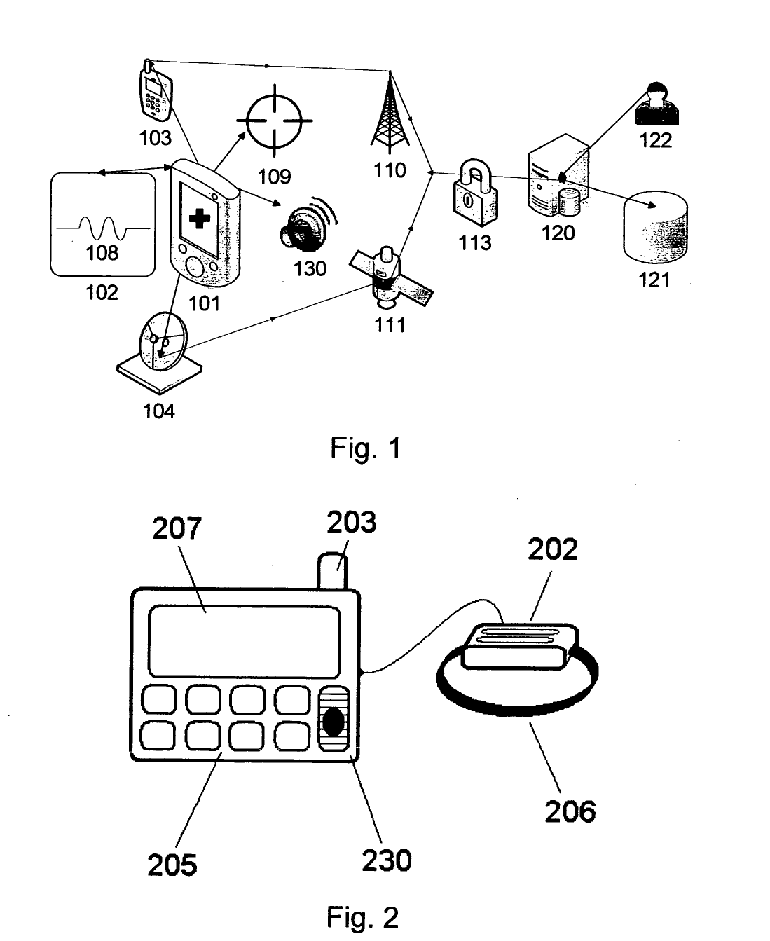

An exemplary embodiment of the present invention is a portable device 101 which reads and transmits medical data through a secure connection 113, as shown in FIG. 1. It works on a system that transmits this medical data through a secure connection 113 between a user and a base server 120 as can be seen in FIG. 1. The secure connection 113 is achieved through a wireless communication device 103,104 either on-board or in communication with the device 101. Radios, cellular telephones 103, or satellite phones 104 can be on-board or in communication with the device 101. The radio or phone 103,104 can be hard-wired into the device 101 or wirelessly connected using a peer to peer protocol such as BLUETOOTH.

The communication device 103,104 then connects to a base server 120. An embodiment using a radio or two-way radio connects to its nearby base server 120 directly to deliver the medical data. An embodiment using a cellular telephone 103 connects to the nearest tower 110 where the signal relays to its specified base server or a global base server 120. Embodiments using a satellite telephone 104 connect to a satellite 111 where the signal relays to its specified base server or a global server 120.

The database 121 stores all of the medical information received by the device 101 and by other sources. These databases 121 can be globally used or locally used. Local databases 121, such as for nursing homes, hospitals, and military groups, have data pertinent to their uses. This database 121 is accessible by the body governing the locality to upload pertinent static information that is necessary, and is updated by these devices 101 with dynamic information 108.

Ideally there would be one global database 121 which stores medical information for everyone, and it would include everything from height and weight to current medication. This static data could be uploaded by anyone with approved access under HIPAA. Individuals can log onto a website to fill in static data such as height, weight, allergies, conditions, etc. Doctor's offices and hospitals can also log onto this website to upload information so that patients do not have to. The database 121 can also be updated by placing a phone call or email message. This database 121 will also receive dynamic data 108 from individuals who choose to have their health constantly monitored and from emergency units while in use.

In addition to the internet, the user has other ways of uploading information to the database 121. On select embodiments, such as shown in FIG. 4, there is a screen 407 and keyboard 405 for entering pertinent information. For personal models that do not feature a screen 407 and keyboard 405 the database 121 can still be accessed via the internet, but for doctors working in the field there may be no computer or internet connection nearby.

There are many detection devices 102 presently that will take a number of medical readings 108. Many of these detection devices 102 come in the form of wristbands 206, as shown in FIG. 2. There are wristbands 206 that will take your pulse, blood pressure, temperature, blood/sugar, or other vital signs 108. Though the wristband 206 is a convenient design, this device 101 is designed to work with any detection device 102 that gives a medical reading 108, and new detection devices 102 are being made everyday. The soldier models will likely have the most thorough medical detection devices 102 connected due to the battlefield medical units need for fast and accurate information 108.

The location feature 109 is also useful on this device 101. Whether on the elderly or specially conditioned, the personal models need locators 109 to tell where the people are in case of emergency. The nursing home models allow the nursing home to keep track of their patients while still allowing them to go where they please and when they please. Field doctors will benefit the most from an on-board GPS locator 109, especially for those who are in third world countries. Soldiers in the battlefield definitely need locators 109 on them. GPS today has an accuracy of within six feet, but depending on the size of the battlefield triangulation might be more accurate. For special operatives, the GPS locator 109 should be preferred.

Voice and data assistance are other benefits of the present invention. The emergency models activate with the push of a button, which immediately begins connecting to the database 121 and an expert 122 who speaks on the line through the on-board speakerphone 130. Before the expert 122 is on the line, on-screen suggestions may appear based on conditions detected, giving simple advice to the user, such as: “keep subject warm”, “elevate subject's feet”, or “turn subject to side”. Personal models, particularly for the elderly and specially conditioned activate by either the push of a button, or upon pre-set conditions met by undesired readings. If these conditions are met, then on-screen tips appear, or an expert 122 will speak through the line in case of emergency. Video may also be added to any of these models for more interactive communication with the expert 122.

An exemplary embodiment of the present invention is the emergency use only embodiment located in buildings and public places as shown in FIG. 2. This embodiment features medical reading attachments 202 for detecting basic vital signs and an on-board cellular telephone 203. Certain features of this embodiment include a main call button among a few other various use buttons 205, and a screen 207. These embodiments can be found behind glass labeled “Break in case of Emergency” and are placed about as commonly as emergency defibrillators. In case of an emergency the glass is broken and this embodiment is removed.

The unit works on a wireless connection 203 and can be carried to the subject, wherever his or her location. Once reached, the wristband 206, or the choice detection device 202 the unit comes with, is placed on the subject and the emergency call button is pressed. The device will immediately check the subject's vital signs 108 while connecting to the database 121 and an expert 122 at the global base 120. While waiting for the expert 122, light on-screen 207 tips are provided in case the rescuer is untrained. Once the expert 122 is summoned he or she will immediately be connected through to the unit at which point the user can communicate through the speakerphone 230. If the expert 122 evaluates a bona fide emergency, then he or she will alert the proper emergency vehicle of the status and location of the subject. The emergency vehicle will most likely be an ambulance, but in the case of some national parks or taller buildings could be a helicopter or fire engine. Once an emergency vehicle is contacted the expert 122 will ask for identification of the subject. If identifiable, the expert 122 can pull up all the medical records of the subject, including allergies, vaccinations, recent complaints or problems, and current medications, which can shed light on the situation and possible treatment options.

Some of the exemplary embodiments according to the present invention are equipped with cellular telephones 203 and GPS locators 109. In the case of some national parks the communication device will be a satellite telephone 104 to ensure a proper connection when needed, no matter where the device is located.

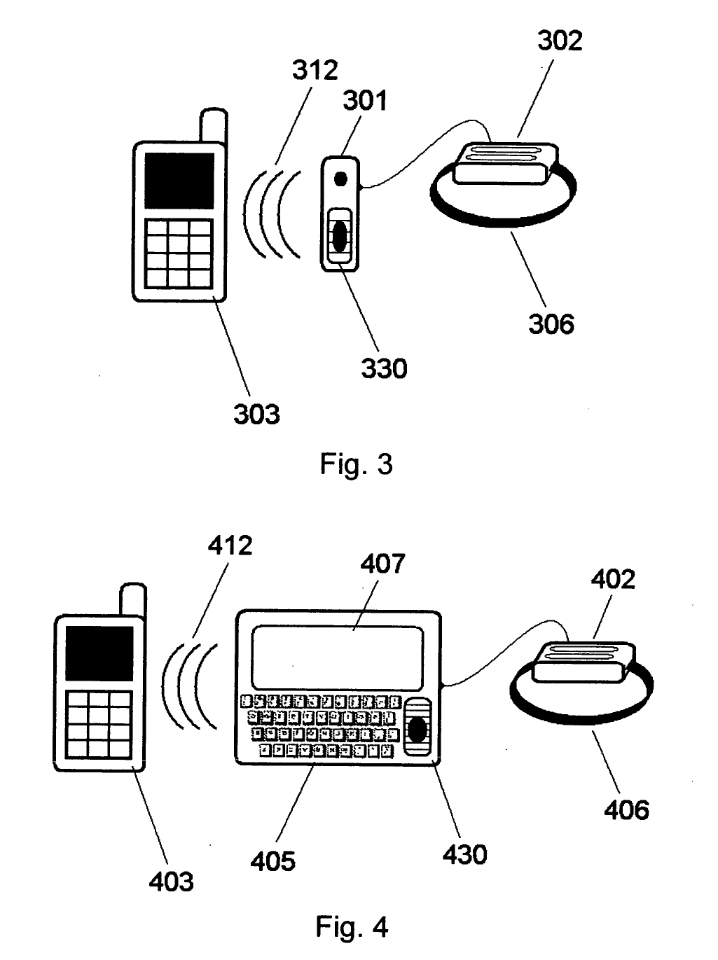

Another exemplary embodiment of the present invention is the personal model for the elderly, specially conditioned, and soldiers as shown in FIG. 3. This range of embodiments features detecting devices 302 specially adapted for the individual user's needs. Diabetics can use this model to take periodic blood/sugar readings while people with hypertension can have their blood pressure checked. Elderly people may have different specific readings performed as well as a basic pulse monitor. A locator 109 may be placed on these embodiments as well.

Conditions are set by the user on these models for a non optimal status. The non optimal status can mean different things to different users. Diabetics can set a non optimal status conditioned upon a low blood/sugar level while someone with hypertension can set their non optimal status conditioned upon a high blood pressure. The user is free to set the status to depend on one reading or a multitude of readings 108, perhaps having an algorithm to determine what constitutes non optimal, or a percentage of the optimal status. Once the non optimal status is reached the user is alerted, either by the communication device 303 or an on-board screen 307. The alert tells the user the non optimal status has been reached and suggests ways to bring the user back to optimal status. These suggestions are more often preset by the user, doctor, or caretaker.

At least one more condition may be set for an emergency status. The emergency status can also mean different things to different users, but some emergency states will be universal, such as a lack of pulse, breathing, blood pressure, or any other condition that is globally recognized as an emergency. User set conditions, such as extremely low blood/sugar or extremely high blood pressure, have the same effect of emergency. Once emergency status is reached this embodiment immediately alerts the base 120 of the status and position, and begins connecting to an expert 122 who will contact the user through the speakerphone 130. If the user is awake, the expert 122 will guide him or her to safety, and to make sure it is not a false alarm. For local models someone will most likely immediately begin searching for the user just in case of an emergency, even if it does turn out to be a false alarm. For global models, the expert 122 may contact the appropriate rescue team once the emergency status is confirmed.

Models of this embodiment that are made for combat soldiers, or special operatives, who are going into battle may require more advanced detection devices 302 connected to the device. The condition will most likely be closely monitored by a local base so a specific indicator of status, such as a percentage, is likely preferred. Depending on the capabilities of the connected detection devices 302, each soldier's status can be viewed in great detail. Soldiers on a battlefield will most likely use a radio, and, depending on the size of the battlefield, may use triangulation to determine each soldier's position rather than relying on GPS. Since triangulation is a job performed by the main receiving towers instead of each individual radio, no extra energy is required by the unit to determine position. Special operatives will most likely prefer a cellular telephone 303 or satellite phone 104 for the communication device. Since their position could be anywhere in the world, a GPS unit is also most likely preferred over triangulation. These soldier embodiments usually do not require a screen, any input, or a speakerphone 130.

Yet another embodiment of the present invention is a fully-loaded, completely functional model made with the field doctor in mind as shown in FIG. 4. This embodiment is designed to be a workstation for users in locations ranging from the sideline of a football field to a third-world country. It comes either as an all-in-one unit or can connect to the user's radio, cellular telephone 403, or satellite phone 104. The detection devices can range from the simple wristband 406 detectors 402 to anything a doctor may find apt for the situation.

The data detected is sent through the secure connection 113 as with the other embodiments, but in this embodiment the user has the option of entering any other data through the input 405, provided the user is a doctor or another privileged person. The input will most likely be a full keyboard 405, but can be more or less. The embodiment can come loaded with only the necessary software to connect to and update the database 121, but can have a plurality of software, including artificial intelligence, to aid the user in tasks that, while difficult, do not require an online expert 122. The user can also look up any facts on the database 121 through this embodiment, and can still request the help of an expert 122 with the push of a button.

Location 109 may not be a concern for every user of this embodiment, but GPS is more likely preferred by most users, especially doctors in third-world countries. The doctor or trainer on the sideline of the football field can probably use a simple radio in connection with this embodiment, but most users will likely prefer a cellular telephone 103. Doctors in third-world countries can probably benefit the most from a satellite phone 104 to make sure they will have a connection whenever necessary.

In the exemplary embodiments described above, all wireless lines of communication may be separate from conventional wireless lines in frequency or other variable so as to separate the line from conventional use. In other words, all wireless signals for the present invention may be made through a separate and distinct pathway, particularly made secure because of the strictly proprietary and confidential (medical) information that may be transmitted therethrough. Such secure and separate line may be made to comply strictly with HIPAA guidelines and have extraordinary precautions and safeguards, such as multiple encryption and other techniques. Thus, a new pathway of communication specifically designed for medical information is within the purview of the present invention so as to ensure the privacy and integrity of the information being transmitted through the embodiments of the present invention described above.

Furthermore, the use of the present invention in its various embodiments described above are not specifically restricted to medical uses. Such a device can also be used to transmit data from a source to a central database for storage or feedback. In one non-limiting example, a user may use a device such as the ones described above to input calorie intake instantaneously into the device for transmission to a central database that accounts for the daily calorie intake for the user, such as for conventional calorie counting diet programs, like WEIGHT WATCHERS and others. Many other uses are also possible and within the purview of the present invention.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A portable communication device comprising:
a central portion capable of receiving medical readings and human input and delivering medical output; and
a communication portion in communication with the central portion capable of sending and receiving data between the device and a server;
wherein the data is sent to the server through a secure connection.

2. The device in claim 1, wherein the communication portion is a radio, cellular telephone, or satellite phone.

3. The device in claim 1, where medical readings are sent to the central portion from a single or plurality of detection devices.

4. The device in claim 1, wherein the data exchanged between the device and the server can be medical readings, medical information, a voice call, or a video call.

5. The device in claim 1, wherein the device is capable of sending a location.

6. The device in claim 5, wherein the device sends a location read by an attached GPS locator unit.

7. The device in claim 1, wherein the server is capable of determining a location using triangulation.

8. The device in claim 1, wherein the detection device or devices are capable of reading pulse, temperature, blood pressure, blood/sugar, or chest movement.

9. The device in claim 1, wherein the server maintains a database of medical information.

10. The server in claim 9, wherein the database can be updated via input from the central portion or a secure connection from another phone, website, or email.

11. A method of sharing medical information comprising steps of:
receiving medical data and transferring it to a communication device;
sending the medical data through a secure connection to a server for storage; and
recalling the medical data and sending it through the secure connection to a recipient.

12. The method of claim 11, wherein the medical data includes an individual's height, weight, allergies, diseases, vaccinations, current medication, pulse, temperature, blood pressure, blood/sugar, or chest movement.

13. The method of claim 11, wherein the medical data is detected from a detection device or a plurality of detection devices.

14. The method of claim 11, wherein the communication device is a radio, cellular telephone, or satellite phone.

15. The method of claim 11, wherein the connection is secured by encryption.

16. The method of claim 11, wherein the server stores the medical data in a database.

17. The server of claim 16, wherein the database contains medical data about a plurality of individuals.

18. The method of claim 16, wherein the medical data is recalled by an expert.

19. The method of claim 11, wherein the recipient is a doctor, nurse, health professional, or other recipient approved by HIPAA.

20. A method of sharing medical information comprising steps of:
receiving medical data from a detection device or plurality of devices and transferring it to a communication device;
sending the medical data through a secure connection to a server for storage; and
recalling the medical data and sending it through the secure connection to a recipient.
